SEQUENCE LISTING (4 sequences)

SEQ ID NO: 1 - protein, 183 aa, Homo sapiens
GPVPTPPDNIQVQENFNISRIYGKWYNLAIGSTCPWLKKIMDRMTVSTLVLGEGATEAEISMTSTRWRKGVCEETSGAYEKTDTDGKFLYHKSKWNITMESYVVHTNYDEYAIFLTKKFSRHHGPTITAKLYGRAPQLRETLLQDFRVVAQGVGIPEDSIFTMADRGECVPGEQEPEPILIPR

SEQ ID NO: 2 - protein, 201 aa, Homo sapiens
MHHHHHHHHGGGGGIEGRGPVPTPPDNIQVQENFNISRIYGKWYNLAIGSTCPWLKKIMDRMTVSTLVLGEGATEAEISMTSTRWRKGVCEETSGAYEKTDTDGKFLYHKSKWNITMESYVVHTNYDEYAIFLTKKFSRHHGPTITAKLYGRAPQLRETLLQDFRVVAQGVGIPEDSIFTMADRGECVPGEQEPEPILIPR

SEQ ID NO: 3 - DNA, 549 bp, Homo sapiens
ggccctgtgccaacgccgcccgacaacatccaagtgcaggaaaacttcaatatctctcggatctatgggaagtggtacaacctggccatcggttccacctgcccctggctgaagaagatcatggacaggatgacagtgagcacgctggtgctgggagagggcgctacagaggcggagatcagcatgaccagcactcgttggcggaaaggtgtctgtgaggagacgtctggagcttatgagaaaacagatactgatgggaggtttctctatcacaaatccaaatggaacataaccatggagtcctatgtggtccacaccacctatgatgagtatgccatttttctgaccaagaaattcagccgccatcatggacccaccattactgccaagctctacgggcgggcgccgcagctgagggaaactctcctgcaggacttcagagtggttgcccagggtgtgggcatccctgaggactccatcttcaccatggctgaccgaggtgaatgtgtccctggggagcaggaaccagagcccatcttaatcccgaga

SEQ ID NO: 4 - DNA, 603 bp, Homo sapiens
atgcatcaccatcaccatcaccatcacggtggaggagggggtatcgagggccgcggccctgtgccaacgccgcccgacaacatccaagtgcaggaaaacttcaatatctctcggatctatgggaagtggtacaacctggccatcggttccacctgcccctggctgaagaagatcatggacaggatgacagtgagcacgctggtgctgggagagggcgctacagaggcggagatcagcatgaccagcactcgttggcggaaaggtgtctgtgaggagacgtctggagcttatgagaaaacagatactgatgggaggtttctctatcacaaatccaaatggaacataaccatggagtcctatgtggtccacaccacctatgatgagtatgccatttttctgaccaagaaattcagccgccatcatggacccaccattactgccaagctctacgggcgggcgccgcagctgagggaaactctcctgcaggacttcagagtggttgcccagggtgtgggcatccctgaggactccatcttcaccatggctgaccgaggtgaatgtgtccctggggagcaggaaccagagcccatcttaatcccgaga

(19) United States
(12) Patent Application Publication
(10) Pub. No.: US 2019/0054142 A1
(43) Pub. Date: Feb. 21, 2019

(54) ALPHA-1-MICROGLOBULIN FOR USE IN THE PROTECTION OF KIDNEYS IN CONNECTION WITH USE OF CONTRAST MEDIA

(71) Applicant: A1M Pharma AB, Lund (SE)

(72) Inventors: Martin AUSTIN, Riehen (CH); Magnus Göran GRAM, Oxie (SE); Bo ÅKERSTRÖM, Lund (SE)

(73) Assignee: A1M Pharma AB, Lund. (SE)

(21) Appl. No.: 16/079,787

(22) PCT Filed: Feb. 24, 2017
(86) PCT No.: PCT/EP2017/054349

§ 371 Date: Aug. 24, 2018

(30) Foreign Application Priority Data
Feb. 25, 2016 (DK) PA 2016 70104

(51) Int. Cl. A61K 38/17; A61K 51/08

(52) U.S. Cl. CPC A61K 38/1722; A61K 51/08

(57) ABSTRACT

This invention relates to an alpha-1-microglobulin for use in prevention of contras medium nephropathy.

### FIELD OF THE INVENTION

The present invention relates to the use of alpha-1-microglobulin (A1M) to prevent contrast media-induced nephropathy (contrast-induced nephropathy including other kidney-associated side-effects, and abbreviated as CIN) or to alleviate or treat nephropathy associated with the use of contrast media.

### BACKGROUND OF THE INVENTION

CIN is one of the leading causes of hospital-acquired acute renal failure. It is associated with a significant higher risk of in-hospital and 1-year mortality, even in patients who do not need dialysis. In fact CIN is the third most common cause of all hospital-acquired acute renal failure and accounts for approximately 10% of all cases.

Today, there is no standard way to define CIN. However, in general CIN can be defined as the impairment of renal function or acute kidney injury occurring within 48 hours after administration of contrast material. The observation of contrast-induced nephrophathy may rely on serial plasma creatinine concentrations. A baseline level should be obtained before administration of the contrast medium, and normally, any negative influence on the kidney is observed if there is a 25% increase in plasma creatinine (SCr) from baseline, or a 0.5 mg/dL (44 micromol/L) increase in SCr from absolute value is seen.

At present the best therapy for CIN is prevention eg by use of as little contrast medium as necessary, withdrawal of nephrotoxic drugs (eg NSAIDs, aminoglycosides, amphothericin B, cyclosporine, tacrolimus) at least 24 hours before administration of contrast medium, and other drugs like eg metformin and ACE-inhibitors should also be withdrawn before administration of contrast medium. An effective means of preventing CIN seems to be hydration therapy although no randomized, controlled trial seems to have been performed today. Fluids with different compositions and tonicity have been studied and normal saline was found to be superior when administered intravenously.

A number of different treatment regimens for CIN, once developed, have been tested including the use of statins, bicarbonate, N-acetylcysteine, ascorbic acid, theophylline, aminophylline, vasodilators, forced diuretics and renal replacement therapy.

However, presently there is still a need for developing effective means for preventing NIC and/or for treating NIC. However, according to the applicant's understanding the mechanisms behind NIC are not fully understood.

### DESCRIPTION OF THE INVENTION

Contrast media are chemical substances used in medical X-ray, magnetic resonance (MRI), computed tomography (CT), angiography and ultrasound imaging. Contrast media enhance and improve the quality of images or pictures so that a radiologist more accurately can reveal any disease or abnormality in the body investigated. In the present context a contrast medium is intended for medical use, ie to be administered to a human who is subject to investigation of the body or part of the body.

Contrast media are used in many different applications including inter alias
angiocardiography (eg ventriculography, selective coronary arteriography),
angiography (eg coronary angiography),
aortography including studies of the aortic root, aortic arch, ascending aorta, abdominal aorta and its branches,
arteriography,
arthography,
contrast enhancement for computed tomographic head and body imaging,
intravenous digital subtraction angiography of the head, neck, abdominal, renal and peripheral vessels,
gastrointestinal studies (eg pass-thru examination of the gastrointestinal tract,
lumbar epidural venograms
nephroangiography
peripheral arteriography,
ventriculaography,
urography (eg excretory urography)
Etc.

Common contrast media include iodine contrast media such as diatriazole (as meglumine or sodium); ioxithalamate; ioxaglate iohexol; iopamidol; iomeprol, ioversol, iopromide, iodixanol, iotrolan, and gadolinium (Gd) contrast media. About a dozen different Gd-chelated agents have been approved as MRI contrast agents around the world.

As a free ion, gadolinium is reported often to be highly toxic, but MRI contrast agents are chelated compounds and are considered safe enough to be used in most persons. The toxicity of free gadolinium ions in animals is due to interference with a number of calcium-ion channel dependent processes. The 50% lethal dose is about 100-200 mg/kg. No prolonged toxicities have been reported following low dose exposure to gadolinium ions. Toxicity studies in rodents, however show that chelation of gadolinium (which also improves its solubility) decreases its toxicity with regard to the free ion by at least a factor of 100 (i.e., the lethal dose for the Gd-chelate increases by 100 times) It is believed therefore that clinical toxicity of Gd contrast agents in humans will depend on the strength of the chelating agent; however this research is still not complete.

Gadolinium MRI contrast agents have proved safer than the iodinated contrast agents used in X-ray radiography or computed tomography. Anaphylactoid reactions are rare, occurring in approximately 0.03-0.1%.

Although gadolinium agents have proved useful for patients with renal impairment, in patients with severe renal failure requiring dialysis, there is a risk of a rare but serious illnesses, called nephrogenic systemic fibrosis (NSF) or nephrogenic fibrosing dermopathy, which has been linked to the use of four gadolinium-containing MRI contrast agents. The disease resembles scleromyxedema and to some extent scleroderma. It may occur months after contrast has been injected. Its association with gadolinium and not the carrier molecule is confirmed by its occurrence in from contrast materials in which gadolinium is carried by very different carrier molecules. NSF is rare and, so far, has only occurred in people with severe kidney disease. Thus, if a severe kidney disease can be prevented or treated in patients undergoing contrast medium investigation, diseases like NSF can be prevented, treated or alleviated.

Current guidelines in the United States suggest that dialysis patients should only receive gadolinium agents where essential and to consider performing an iodinated contrast enhanced CT when feasible. If a contrast enhanced MRI must be performed on a dialysis patient, it is recommended that certain high-risk contrast agents be avoided and that a lower dose be considered. The American College of Radiology recommends that contrast enhanced MRI examinations be performed as closely before dialysis as possible as a precautionary measure, although this has not been proven to reduce the likelihood of developing NSF.

Contrast media are normally classified. It is believed that CIN is influenced by the osmolality of the contrast medium and, accordingly, a contrast medium may be a low-osmolarity contrast medium (LOCM) or a high-osmolarity contrast medium (HOCM). In the present context, all medical contrast media are of relevance, in particular those with known CIN effects.

Other contrast media includes radio-isotopes like 68-gallium, 111-indium, 99m-technetium, 201-thallium, fludeoxyglucose (18F-FDG), 18-flourine, 131-iodine, 60-cobalt and the like.

Not encompassed by the present invention is the use of A1M in combination with radionuclide diagnostics, radionuclide therapy or radioimmunotherapy. Especially, in those cases where the radionuclide is a somatostatin-analogous peptide labelled with a therapeutic radionuclide.

The present invention relates to A1M for use in prevention of kidney damages generally resulting after administration of a contrast medium to a patient. The invention also relates to the treatment of such kidney damages, wherein the treatment is initiated before, during or after the administration of a contrast medium to a patient.

In particular, the invention relates to the use of A1M for prevention of kidney damages, notably or specifically in a subpopulation of patients undergoing investigation with a contrast medium, wherein the subpopulation comprises patients or persons with one or more of the following risk factors:
Age—increasing age leads to increasing risk, normally 75 years or more is regarded as a critical factor,
CKD (chronic kidney disease)
Diabetes mellitus
Hypertension
Metabolic syndrome
Anemia
Multiple myeloma
Hypoalbuminemia
Renal transplant
Hypovolemia and decreased effective circulating volumes as evidenced by
   Congestive heart failure (CHF)
   Ejection fraction (EF) of less than 40%
   Hypotension
   Intra-aortic balloon counterpulsation

In those cases where a person has one or more of the above-mentioned risk factors, it would be advantageous to administer A1M to the person undergoing body part inspection via use of a contrast medium. A1M may be given before, essentially at the same time, during or after administration of the contrast medium.

Other important factors involving in risk assessment are:
Urgent versus elective (urgent being more risky than elective)
Arterial versus venous (arterial being more risky than venous)
Diagnostic versus therapeutic (diagnostic being more risky than therapeutic).

Contrast-related risk factors are:
Volume of contrast (larger volumes are more risky)
Contrast characteristics, including osmolarity, tonicity, molecular structure and viscosity (studies indicate that use of isoosmolar contrast medium is less risky than hypo- or hyperosmolar contrast media)

The single most important patient-related risk factor is preexisting CKD associated with diabetes mellitus. Patients with CKD in the setting of diabetes mellitus have a 4-fold increase in the risk of CIN compared with patients without diabetes mellitus or preexisting CKD.

Moreover, the invention relates to ai-microglobulin (A1M) for use in the treatment of kidney-associated side-effects resulting from use of contrast medium in a human, wherein A1M is used as a co-treatment to the contrast medium used. A1M may be administered essentially at the same time as the contrast medium, or A1M therapy may be initiated once the kidney-associated side-effects appear or is evident based on patient monitoring eg of relevant kidney function parameters such as creatinine of eGFR (estimated glomerular filtration rate), cystatin C or other reliable markers of kidney function.

A prerequisite for a protective action of A1M is of course that the protein is 1) localized to the kidneys after exogeneous administration, 2) not degraded immediately after its localization in the kidneys. We therefore investigated the 1) kinetics of biodistribution of infused A1M, and 2) size of the protein in kidney homogenates. As evident from FIG. 1, the major part of infused A1M is localized to the kidneys after 10 min. FIG. 2 shows that the majority of A1M found in the kidneys display full-length size at least up to 60 minutes post-injection. A natural route of A1M in the kidneys, similar to most small plasma proteins, is glomerular filtration from blood to the primary urine, followed by reabsorption and lysosomal degradation in the proximal tubular epithelium [29,30]. A small fraction of A1M can still be found in urine [24]. It can therefore be speculated that although a large part is expected to be degraded in the proximal tubular cells, a significant amount of A1M may escape tubular reabsorption and degradation and is left intact and functional during the first 10-60 min.

### α₁-Microglobulin—a General Background

A1M is synthesized in the liver at a high rate, secreted into the blood stream and transported across the vessel walls to the extravascular compartment of all organs. The protein is also synthesized in other tissues (blood cells, brain, kidney, skin) but at a lower rate. Due to the small size, free A1M is rapidly filtered from blood in the kidneys.

A1M is a member of the lipocalin superfamily, a group of proteins from animals, plants and bacteria with a conserved three-dimensional structure but very diverse functions. Each lipocalin consists of a 160-190-amino acid chain that is folded into a β-barrel pocket with a hydrophobic interior. At least twelve human lipocalin genes are known. A1M is a 26 kDa plasma and tissue protein that so far has been identified in mammals, birds, fish and frogs. The three-dimensional structure of A1M determined by X-ray crystallography is shown in FIG. 3. A1M is synthesized in the liver at a high rate, secreted into the blood stream and rapidly (T½=2-3 min) transported across the vessel walls to the extravascular compartment of all organs. A1M is found both in a free, monomeric form and as covalent complexes with larger molecules (IgA, albumin, prothrombin) in blood and interstitial tissues. Due to the small size, free A1M is rapidly filtered from blood in the kidneys. The major portion is then reabsorbed, but significant amounts are excreted to the urine.

Antioxidants are protective factors that eliminate oxidants or prevent harmful oxidation reactions. The human organism can produce antioxidants in response to oxidative stress. Such endogenous antioxidants include the superoxide-degrading enzyme superoxide dismutase (SOD), the hydrogen peroxide-degrading enzymes catalase and glutathione peroxidase, and the heme-degrading enzyme heme oxygenase-1 (HO-1). A normally occurring 26 kDa plasma and tissue protein, α₁-microglobulin (A1M), was recently shown to be involved in protecting against oxidative tissue damage by functioning both as a scavenger of radicals and heme as well as a reductase and inhibitor of oxidation. Several recent papers demonstrate that A1M protects cell cultures and organ explants against oxidative damage, partly by accumulating in mitochondria and protecting mitochondrial function. Indeed, infusion of human recombinant A1M has been successfully employed for in vivo treatment of the oxidative stress-related diseases preeclampsia and hemoglobin-induced glomerular injuries in animal models.

### Sequence and Structural Properties of A1M

The full sequence of human A1M is known. The protein consists of a polypeptide with 183 amino acid residues. Many additional A1M cDNAs and/or proteins have been detected, isolated and/or sequenced from other mammals, birds, amphibians, and fish. The length of the peptide chain of A1M differs slightly among species, due mainly to variations in the C-terminus. Alignment comparisons of the different deduced amino acid sequences show that the percentage of identity varies from approximately 75-80% between rodents or ferungulates and man, down to approximately 45% between fish and mammals. A free cysteine side-chain at position 34 is conserved. This group has been shown to be involved in redox reactions (see below), in complex formation with other plasma proteins and in binding to a yellow-brown chromophore. The three-dimensional structure of A1M shows that C34 is solvent exposed and located near the opening of the lipocalin pocket (see FIG. 3).

In the present context the term “α₁-microglobulin” intends to cover α₁-microglobulin as identified in SEQ ID NO: 1 (human A1M) as well as SEQ ID NO: 2 (human recombinant A1M) as well as homologues, fragments or variants thereof having similar therapeutic activities. Thus, A1M as used herein is intended to mean a protein having at least 80% sequence identity with SEQ ID NO:1 or SEQ ID NO:2. It is preferred that A1M as used herein has at least 90% sequence identity with SEQ ID NO:1 or SEQ ID NO:2. It is even more preferred that A1M as used herein has at least 95% such as 99% or 100% sequence identity with SEQ ID NO:1 or SEQ ID NO:2. In a preferred aspect, the α₁-microglobulin is in accordance with SEQ ID NO: 1 or 2 as identified herein. In FIG. 7 is given the sequence listing of the amino acid sequence of human A1M and human recombinant A1M (SEQ ID NOs 1 and 2, respectively) and the corresponding nucleotide sequences (SEQ ID NOs 3 and 4, respectively). However, homologues, variants and fragments of A1M having the important parts of the proteins as identified in the following are also comprised in the term A1M as used herein.

As mentioned above homologues of A1M can also be used in accordance with the description herein. In theory A1M from all species can be used for the purposes described herein including the most primitive found so far, which is from fish (plaice). A1M is also available in isolated form from human, orangutan, squirrel monkey, rat, naked mole rat, mouse, rabbit, guinea pig, cow, frog, chicken, walrus, manatee and plaice.

Considering homologues, variants and fragments of A1M, the following has been identified as important parts of the protein for the anti-oxidative effect:

Y22 (Tyrosine, pos 22, basepairs 64-66)
C34 (Cystein, position 34, basepairs 100-102)
K69 (Lysine, pos 69, basepairs 205-207)
K92 (Lysine, pos 92, basepairs 274-276)
K118 (Lysine, pos 118, basepairs 352-354)
K130 (Lysine, pos 130, basepairs 388-390)
Y132 (Tyrosine, pos 132, basepairs 394-396)
L180 (Leucine, pos 180, basepairs 538-540)
1181 (Isoleucine, pos 181, basepairs 541-543)
P182 (Proline, pos 182, basepairs 544-546)
R183 (Arginine, pos 183, basepairs 547-549)

(Numbering of amino acids and nucleotides throughout the document refers to SEQ ID 1 and 3, see also FIGS. 3 and 4; if other A1M from other species, A1M analogs or recombinant sequences thereof are employed, a person skilled in the art will know how to identify the amino acids of the active site(s) or site(s) responsible for the enzymatic activity.)

Thus, in those cases, where A1M eg has 80% (or 90% or 95%) sequence identity with one of SEQ ID NO: 1 or 2, it is preferred that the amino acids mentioned above are present at the appropriate places in the molecule.

Human A1M is substituted with oligosaccharides in three positions, two sialylated complex-type, probably diantennary carbohydrated linked to N17 and N96 and one more simple oligosaccharide linked to T5. The carbohydrate content of A1M proteins from different species varies greatly, though, ranging from no glycosylation at all in Xenopus lea vis over a spectrum of different glycosylation patterns. However, one glycosylation site, corresponding to N96 in man, is conserved in mammals, suggesting that this specific carbohydrate may be functionally important.

A1M is yellow-brown-coloured when purified from plasma or urine. The colour is caused by heterogeneous compounds covalently bound to various amino acid side groups mainly located at the entrance to the pocket. These modifications represent the oxidized degradation products of organic oxidants covalently trapped by A1M in vivo, for example heme, kynurenine and tyrosyl radicals.

A1M is also charge- and size-heterogeneous and more highly brown-coloured A1M-molecules are more negatively charged. The probable explanation for the heterogeneity is that different side-groups are modified to a varying degree with different radicals, and that the modifications alter the net charge of the protein. Covalently linked coloured substances have been localized to C34, and K92, K118 and K130, the latter with molecular masses between 100 and 300 Da. The tryptophan metabolite kynurenine was found covalently attached to lysyl residues in A1M from urine of haemodialysis patients and appears to be the source of the brown colour of the protein in this case [6]. Oxidized fragments of the synthetic radical ABTS (2,2″-azino-di-(3-ethylbenzothiazoline)-6-sulfonic acid) was bound to the side-chains of Y22 and Y132.

C34 is the reactive center of A1M. It becomes very electronegative, meaning that it has a high potential to give away electrons, by the proximity of the positively charged side-chains of K69, K92, K118 and K130, which induce a deprotonization of the C34 thiol group which is a prerequisite of oxidation of the sulphur atom. Preliminary data shows that C34 is one of the most electronegative groups known.

Theoretically, the amino acids that characterize the properties of A1M (C34, Y22, K92, K118, K130, Y132, L180, 1181, P182, R183), which will be described in more detail below, can be arranged in a similar three-dimensional configuration on another framework, for instance a protein with the same global folding (another lipocalin) or a completely artificial organic or inorganic molecule such as a plastic polymer, a nanoparticle or metal polymer.

The three-dimensional arrangement of some of these amino acids (blue ovals, the lysines are depicted by a “+”), the A1M-framework (barrel), the electron-flow and the radical-trapping, are illustrated in FIG. 4.

Accordingly, homologues, fragments or variants comprising a structure including the reactive centre and its surroundings as depicted above, are preferred.

Modifications and changes can be made in the structure of the polypeptides of this disclosure and still result in a molecule having similar characteristics as the polypeptide (e.g., a conservative amino acid substitution). For example, certain amino acids can be substituted for other amino acids in a sequence without appreciable loss of activity. Because it is the interactive capacity and nature of a polypeptide that defines that polypeptide's biological functional activity, certain amino acid sequence substitutions can be made in a polypeptide sequence and nevertheless obtain a polypeptide with like properties.

In making such changes, the hydropathic index of amino acids can be considered. The importance of the hydropathic amino acid index in conferring interactive biologic function on a polypeptide is generally understood in the art. It is known that certain amino acids can be substituted for other amino acids having a similar hydropathic index or score and still result in a polypeptide with similar biological activity. Each amino acid has been assigned a hydropathic index on the basis of its hydrophobicity and charge characteristics. Those indices are: isoleucine (+4.5); valine (+4.2); leucine (+3.8); phenylalanine (+2.8); cysteine/cysteine (+2.5); methionine (+1.9); alanine (+1.8); glycine (0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is believed that the relative hydropathic character of the amino acid determines the secondary structure of the resultant polypeptide, which in turn defines the interaction of the polypeptide with other molecules, such as enzymes, substrates, receptors, antibodies, antigens, and the like. It is known in the art that an amino acid can be substituted by another amino acid having a similar hydropathic index and still obtain a functionally equivalent polypeptide. In such changes, the substitution of amino acids whose hydropathic indices are within ±2 is preferred, those within ±1 are particularly preferred, and those within ±0.5 are even more particularly preferred.

Substitution of like amino acids can also be made on the basis of hydrophilicity, particularly where the biologically functional equivalent polypeptide or peptide thereby created is intended for use in immunological embodiments. The following hydrophilicity values have been assigned to amino acid residues: arginine (+3.0); lysine (+3.0); aspartate (+3.0±1); glutamate (+3.0±1); serine (+0.3); asparagine (+0.2); glutamnine (+0.2); glycine (0); proline (−0.5±1); threonine (−0.4); alanine (−0.5); histidine (−0.5); cysteine (1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent polypeptide. In such changes, the substitution of amino acids the hydrophilicity values of which are within ±2 is preferred, those within ±1 are particularly preferred, and those within ±0.5 are even more particularly preferred.

As outlined above, amino acid substitutions are generally based on the relative similarity of the amino acid side-chain substituents, for example, their hydrophobicity, hydrophilicity, charge, size, and the like. Exemplary substitutions that take one or more of the foregoing characteristics into consideration are well known to those of skill in the art and include, but are not limited to (original residue: exemplary substitution): (Ala: Gly, Ser), (Arg: Lys), (Asn: Gln₁ His), (Asp: Glu, Cys, Ser), (Gln: Asn), (Glu: Asp), (Gly: Ala), (His: Asn, Gln), (Ile: Leu, Val), (Leu: Ile, Val), (Lys: Arg), (Met: Leu, Tyr), (Ser: Thr), (Thr: Ser), (Trp: Tyr), (Tyr: Trp, Phe), and (Val: Lle, Leu). Embodiments of this disclosure thus contemplate functional or biological equivalents of a polypeptide as set forth above. In particular, embodiments of the polypeptides can include variants having about 50%, 60%, 70%, 80%, 90%, and 95% sequence identity to the polypeptide of interest.

In the present context, the homology between two amino acid sequences or between two nucleic acid sequences is described by the parameter “identity”. Alignments of sequences and calculation of homology scores may be done using a full Smith-Waterman alignment, useful for both protein and DNA alignments. The default scoring matrices BLOSUM50 and the identity matrix are used for protein and DNA alignments respectively. The penalty for the first residue in a gap is −12 for proteins and −16 for DNA, while the penalty for additional residues in a gap is −2 for proteins and −4 for DNA. Alignment may be made with the FASTA package version v20u6.

Multiple alignments of protein sequences may be made using “ClustalW”. Multiple alignments of DNA sequences may be done using the protein alignment as a template, replacing the amino acids with the corresponding codon from the DNA sequence.

Alternatively different software can be used for aligning amino acid sequences and DNA sequences. The alignment of two amino acid sequences is e.g. determined by using the Needle program from the EMBOSS package (http://emboss.org) version 2.8.0. The Needle program implements the global alignment algorithm described in. The substitution matrix used is BLOSUM62, gap opening penalty is 10, and gap extension penalty is 0.5.

The degree of identity between an amino acid sequence; e.g. SEQ ID NO: 1 and a different amino acid sequence (e.g. SEQ ID NO: 2) is calculated as the number of exact matches in an alignment of the two sequences, divided by the length of the “SEQ ID NO: 1” or the length of the “SEQ ID NO: 2”, whichever is the shortest. The result is expressed in percent identity.

An exact match occurs when the two sequences have identical amino acid residues in the same positions of the overlap.

If relevant, the degree of identity between two nucleotide sequences can be determined by the Wilbur-Lipman method using the LASER-GENE™ MEGALIGN™ software (DNASTAR, Inc., Madison, Wis.) with an identity table and the following multiple alignment parameters: Gap penalty of 10 and gap length penalty of 10. Pairwise alignment parameters are Ktuple=3, gap penalty=3, and windows=20.

The percentage of identity of an amino acid sequence of a polypeptide with, or to, amino acids of SEQ ID NO: 1 may be determined by i) aligning the two amino acid sequences using the Needle program, with the BLOSUM62 substitution matrix, a gap opening penalty of 10, and a gap extension penalty of 0.5; ii) counting the number of exact matches in the alignment; iii) dividing the number of exact matches by the length of the shortest of the two amino acid sequences, and iv) converting the result of the division of iii) into percentage. The percentage of identity to, or with, other sequences of the invention is calculated in an analogous way.

By way of example, a polypeptide sequence may be identical to the reference sequence, that is be 100% identical, or it may include up to a certain integer number of amino acid alterations as compared to the reference sequence such that the % identity is less than 100%. Such alterations are selected from: at least one amino acid deletion, substitution (including conservative and non-conservative substitution), or insertion, and wherein said alterations may occur at the amino- or carboxy-terminus positions of the reference polypeptide sequence or anywhere between those terminal positions, interspersed either individually among the amino acids in the reference sequence, or in one or more contiguous groups within the reference sequence.

Conservative amino acid variants can also comprise non-naturally occurring amino acid residues. Non-naturally occurring amino acids include, without limitation, trans-3-methylproline, 2,4-methanoproline, cis-4-hydroxyproline, trans-4-hydroxyproline, N-methyl-glycine, allo-threonine, methylthreonine, hydroxy-ethylcysteine, hydroxyethylhomocysteine, nitro-glutamine, homoglutamine, pipecolic acid, thiazolidine carboxylic acid, dehydroproline, 3- and 4-methylpróline, 3,3-dimethylproline, tert-leucine, norvaline, 2-azaphenylalanine, 3-azaphenylalanine, 4-azaphenylalanine, and 4-fluorophenylalanine. Several methods are known in the art for incorporating non-naturally occurring amino acid residues into proteins. For example, an in vitro system can be employed wherein nonsense mutations are suppressed using chemically aminoacylated suppressor tRNAs. Methods for synthesizing amino acids and aminoacylating tRNA are known in the art. Transcription and translation of plasmids containing nonsense mutations is carried out in a cell-free system comprising an E. coli S30 extract and commercially available enzymes and other reagents. Proteins are purified by chromatography. In a second method, translation is carried out in Xenopus oocytes by microinjection of mutated mRNA and chemically aminoacylated suppressor tRNAs. Within a third method, E. coli cells are cultured in the absence of a natural amino acid that is to be replaced (e.g., phenylalanine) and in the presence of the desired non-naturally occurring amino acid(s) (e.g., 2-azaphenylalanine, 3-azaphenylalanine, 4-azaphenylalanine, or 4-fluorophenylalanine). The non-naturally occurring amino acid is incorporated into the protein in place of its natural counterpart. Naturally occurring amino acid residues can be converted to non-naturally occurring species by in vitro chemical modification. Chemical modification can be combined with site-directed mutagenesis to further expand the range of substitutions. Alternative chemical structures providing a 3-dimensional structure sufficient to support the antioxidative properties of A1M may be provided by other technologies e.g. artificial scaffolds, amino-acid substitutions and the like. Furthermore, structures mimicking the active sites of A1M as listed above and depicted in FIGS. 3 and 4 are contemplated as having the same function as A1M.

### Pharmaceutical Compositions and Dosage

The present invention also provides a kit comprising:

i) a pharmaceutical composition comprising a contrast medium, and

ii) a pharmaceutical composition comprising A1M.

The kit is in the form of one package containing the above-mentioned two compositions.

The pharmaceutical composition comprising a contrast medium is typically a composition already on the market.

The pharmaceutical composition comprising A1M (or an analogue, fragment or variant thereof as defined herein) is intended for i.v. administration. Accordingly, A1M can be formulated in a liquid, e.g. in a solution, a dispersion, an emulsion, a suspension etc.

For parenteral use suitable solvents include water, vegetable oils, propylene glycol and organic solvents generally approved for such purposes. In general, a person skilled in the art can find guidance in “Remington's Pharmaceutical Science” edited by Gennaro et al. (Mack Publishing Company), in “Handbook of Pharmaceutical Excipients” edited by Rowe et al. (PhP Press) and in official Monographs (e.g. Ph. Eur. or USP) relating to relevant excipients for specific formulation types and to methods for preparing a specific formulation.

A1M will be administrated in one or several doses in connection to the administration of contrast medium. Preferably, each dose will be administrated i.v. either as a single dose, as a single dose followed by slow infusion during a short time-period up to 60 minutes, or only as a slow infusion during a short time-period up to 60 minutes. The first dose may be administrated at the same time as the contrast medium, or within a period of 0-60 minutes before to 0-30 minutes after injection of the contrast medium. Additional A1M-doses can be added, but may not be necessary, after injection of the contrast medium. Each dose contains an amount of A1M which is related to the bodyweight of the patient: 1-15 mg A1M/kg of the patient.

### LEGENDS TO FIGURES

FIG. 1 shows the biodistribution of ¹²⁵I-A1M (upper left) in normal NMRI mice. Lower left image shows uptake over time in the kidneys. Data are presented as % IA/g from 4 animals±SEM.

FIG. 2 shows the presence of full-length A1M in normal NMRI mice in kidneys and serum at 10, 20 and 60 minutes post-injection. Animals were injected i.v. with 150 μg A1M and blood and kidneys collected at the indicated time-points. The blood was allowed to coagulate and serum separated by centrifugation. One kidney was homogenized in 1 ml PBS and centrifuged. 1 μl serum and 6 μl supernatant from the kidney homogenate were applied to SDS-PAGE, transferred to PVDF-membranes and blotted with anti-A1M. Each lane represents a separate mouse.

FIG. 3 shows the three-dimensional structure of A1M. The illustration was generated using PyMOL [Molinspiration, M. v. (2014)] and coordinates from the crystal structure of human A1M [Meining, W., and Skerra, A. (2012) The crystal structure of human α₁-microglobulin reveals a potential haem-binding site. Biochem J445, 175-182]. β-strands and α-helices are shown in green ribbons. Side-chains of C34, K92, K118, K130 and H123, involved in functional activities of A1M, are shown as green sticks with nitrogen atoms in blue. The four lipocalin loops are labeled #1-#4.

FIG. 4 shows the three-dimensional arrangement of some amino acids (blue ovals, the lysines are depicted by a “+”), the A1M-framework (barrel), the electron-flow and the radical-trapping.

FIG. 5 shows qualitative SPECT/CT analysis for ¹²⁵I-A1M and visualizes a predominat activity distribution in the kidney cortex, seen from sagittal and dorsal views. A slight uptake of ¹²⁵I-A1M in the thyroids can be seen as well.

FIG. 6 shows the distribution of A1M immunoreactivity in the kidney 20 minutes after i.v. injection. A1M was injected i.v., animals were terminated after 20 minutes, and A1M immunoreactivity was detected with the K323 anti-A1M antibody, using immunohistochemistry. The left panel shows representative areas with A1M-immunoreactivity in the cortex (A), medulla (B), and collecting ducts (C); the location of these areas is indicated with A-C and highlighted with boxes in the schematic drawing in the right panel. Scale bar represents 100 μm in A-C.

FIG. 7 shows the sequences SEQ ID 1-4.

### EXPERIMENTAL

### Materials and Methods

### Recombinant Human A1M

Recombinant human A1M was expressed in E. coli, purified and re-folded as described by Kwasek et al [25] but with an additional ion-exchange chromatography step. This was performed by applying A1M to a column of DEAE-Sephadex A-50 (GE Healthcare, Uppsala, Sweden) equilibrated with 20 mM Tris-HCl, pH8.0. A1M was eluted with a linear salt gradient (from 20 mM Tris-HCl, pH8.0 to 20 mM Tris-HCl, 0.2 M NaCl) at a flow rate of 1 ml/min. A1M-containing fractions, according to absorbance at 280 nm, were pooled and concentrated.

### ¹²⁵I-Labelling of A1M

Radiolabelling of A1M with ¹²⁵I was done using the chloramine T method [26]. Briefly, A1M and ¹²⁵I (Perkin-Elmer, NEZ033005MC) were mixed in 0.5 M sodium phosphate, pH 7.5 at final concentrations of 1 mg/ml and 10 mCi/ml, respectively. Chloramine T was added to 0.4 mg/ml and allowed to react on ice for 2 minutes, and the reaction was stopped by adding NaHSO₃ to 0.8 mg/ml. Protein-bound iodine was separated from free iodide by gel-chromatography on a Sephadex G-25 column (PD10, GE Healthcare, Buckinghamshire, UK). A specific activity of around 50-200 kBq/μg protein was obtained.

### Animal Studies

All animal experiments were conducted in compliance with the national legislation on laboratory animals' protection and with the approval of the Ethics Committee for Animal Research (Lund University, Sweden). Male and female NMRI normal mice of 6-8 weeks old (Taconic, Ry, Denmark) were used.

### Biodistribution

Biodistribution studies were conducted to determine the pharmacokinetics and biodistribution of ¹²⁵I-A1M. ¹²⁵I-A1M (100 kBq, 1 μg) was administered i.v. through tail vein injection to NMRI mice (n=3 per injected molecule and time point). Animals were termination at 10, 20, 40, 60 minutes post-injection and blood and organs were sampled, weighed and measured in a NaI(Tl) well counter (Wallac Wizard 1480 Wizard, Perkin Elmer). Organ-specific uptake values were calculated as percent injected activity per gram of tissue (% IA/g) or percent injected activity (% IA).

### Western Blotting

SDS-PAGE analysis was performed on kidneys and serum from animals that had been injected i.v. with non-labeled A1M (100 μl/animal, 1.5 mg/ml). Animals were terminated at 10, 20 and 60 minutes post-injection, blood and kidneys were sampled and kidneys were washed and placed in 1 ml PBS. Following mechanical tissue homogenization, tissue was centrifuged at 10,000×g for 10 minutes and supernatant was transferred to a new tube and used for further analysis as describe below. Serum was obtained from the blood samples by centrifugation at 1,000×g for 10 minutes. SDS-PAGE gels were run under reducing conditions and the separated proteins were transferred to polyvinylidene difluoride (PVDF) membranes (Immobilon-P, Millipore, Bedford, Mass., USA) using TransBlot Turbo transfer system (Bio-Rad, Delaware, USA). PVDF membranes were subsequently blocked and incubated overnight with the IgG-fraction of rabbit polyclonal anti-A1M antiserum (K322, 5 μg/ml) as described previously], followed by incubation with Alexa Fluor 647 goat anti-rabbit IgG (diluted 3000×; Molecular Probes). The membranes were developed using a ChemiDoc MP Imaging system (BioRad).

### SPECT Imaging

Animals were anaesthetized with 2% to 3% isoflurane gas (Baxter; Deerfield, Ill., USA) during imaging in the NanoSPECT/CT (Bioscan, Washington D.C., USA). Animals were i.v. injected with approximately 5 MBq of ¹²⁵I-A1M (approximately 30 μg) and imaged 20 m p.i. with the NSP-106 multi-pinhole mouse collimator. For ¹²⁵I imaging energy windows of 20% were centered over the 35 keV photo peak and for ¹¹¹In over the 175 and 241 photo peaks. SPECT data were reconstructed using HiSPECT software (SciVis; Goettingen, Germany). CT imaging was done before each whole-body SPECT.

### Kidney—Sample Preparation and Immunolabeling of A1M

Following i.v. injection of 150 μg A1M (unconjugated) animals were sacrificed after 10, 20, 40, 60 minutes and 4 hours. All time-points were evaluated but only kidneys from 20 minutes and 4 hours, displaying detailed analyses at the cellular level, including laser confocal scanning microscopy and quantitative image analyses, are included. Importantly, all experiments were performed and evaluated on both wild-type and nude mice, and was shown to possess the same labeling pattern. However, only wild-type data are included.

After euthanization, kidneys were removed directly frozen and embedded in Tissue Tec. The tissue blocks were sectioned in a cryostat (Microm, HM 5000M, Walldorf, GmbH), and sections (10 μm) were collected on SuperFrost plus slides (Merck, Darmstadt, Germany). Serial sectioning was performed, collecting 3-4 sections per slide, of which adjacent slides were used for chromogen immunohistochemistry (IHC). Sections were post-fixed in 4% paraformaldehyde (PFA, Sigma, St. Louis, Mo., USA, dissolved in PBS, 0.1 M, pH 7.4) for 15 minutes, and rinsed in PBS two times for 5 minutes.

For labeling of A1M, sections were incubated with 0.03% hydrogen peroxide (H₂O₂, Merck, Darmstadt, Germany) for five minutes for chromogen visualization (IHC), and then incubated with 1% bovine serum albumin (BSA, Sigma, St. Louis, Mo., USA; diluted in PBS) for 30 minutes. Sections were then incubated with rabbit anti-human A1M (K:323, IgG), diluted 1:7500 (in PBS containing 1% BSA, 0.02% Triton X-100 (Sigma, St. Louis, Mo., USA) for 16 hours at 4° C.

The sections were then incubated with goat anti-rabbit IgG conjugated with horseradish peroxidase (HRP, Dako Glostrup, Denmark) for 20 minutes at RT. The immunoreaction was performed via incubation in a diaminobenzidine (DAB) solution containing 0.03% H₂O₂, for 10 minutes at RT. Sections were rinsed in PBS (2×10 minutes) and counterstained with hematoxylin (Mayers, Hematoxylin Mayers Htx Histolab Products AB, Gothenburg, Sweden) followed by dehydration in a graded alcohol series and immersion in 100% Xylene. Sections were mounted and cover slipped in Pertex (Histolab Products AB, Gothenburg, Sweden).

Chromogen single labeled A1M was visualized and digitally documented in a bright-field microscope (Leica DMRE). Digital images were collected with a Leica digital camera (DFC 500). Images used for illustrations were corrected for color balance, brightness and contrast.

### Results

### Biodistribution

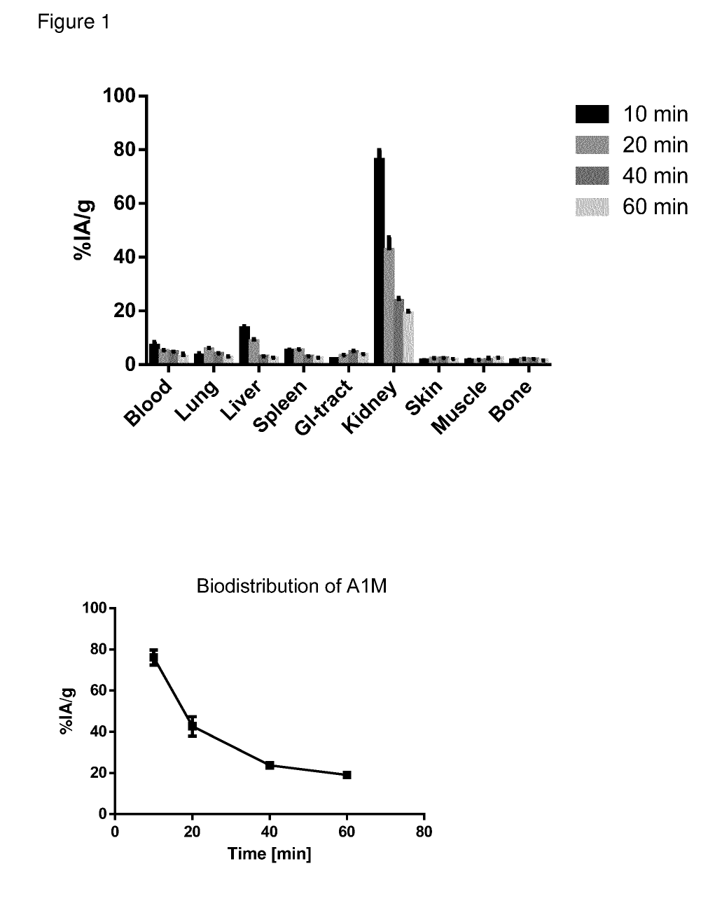

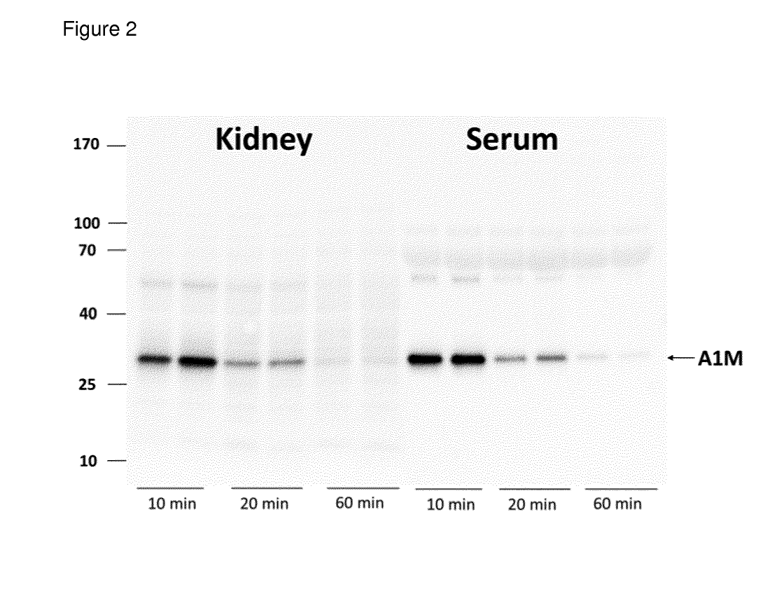

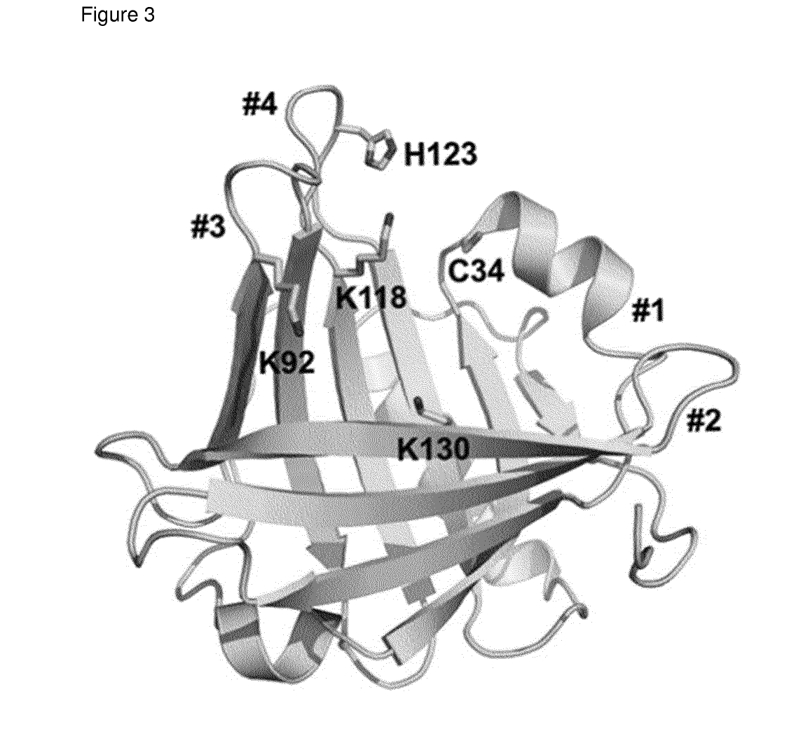

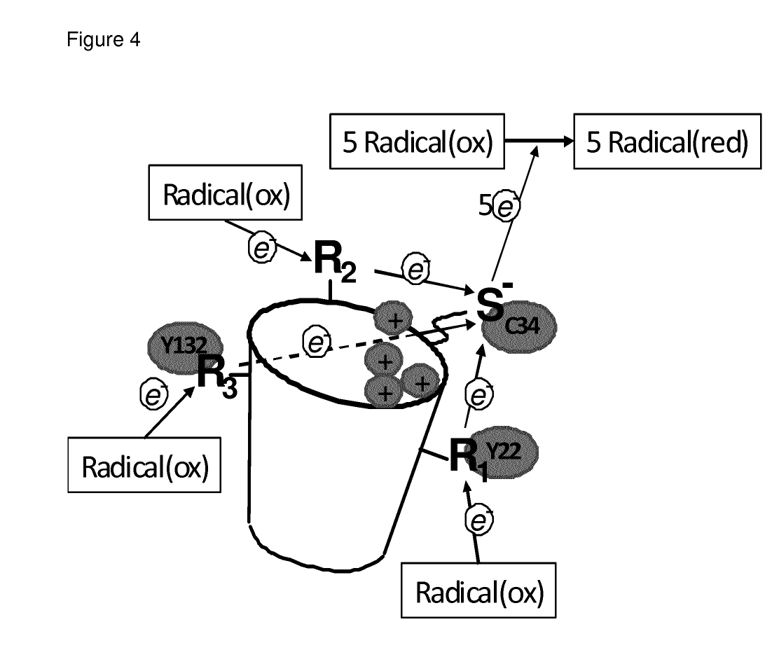

FIG. 1 shows ex vivo biodistribution of ¹²⁵I-A1M at 10, 20, 40 and 60 minutes post-injection as well as 4 and 24 h post injection. High uptake in the kidneys was observed for ¹²⁵I-A1M, with peak value at 10 minutes post-injection. Size distribution of injected non-labelled A1M was investigated in blood serum and solubilized kidneys by SDS-PAGE and Western blotting. As shown in FIG. 2, A1M migrates as a homogeneous band with an apparent molecular mass around 25 kDa both in kidneys and serum at all times, and a minor, faint band around 50 kDa. The strong band most likely represents monomeric A1M with a theoretical molecular mass of 22.6 kDa and the latter the dimeric form. Highest amounts are seen at 10 minutes, supporting the kinetics of ¹²⁵I-labelled A1M shown in FIG. 1, lower panel. These results show that the A1M found in blood and kidneys is intact, full-length and that the degradation therefore is negligible.

### SPECT/CT Image Analysis

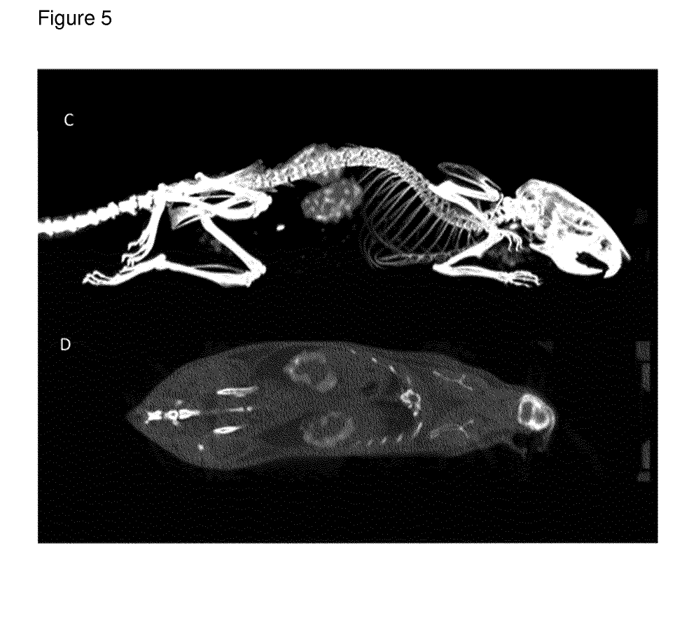

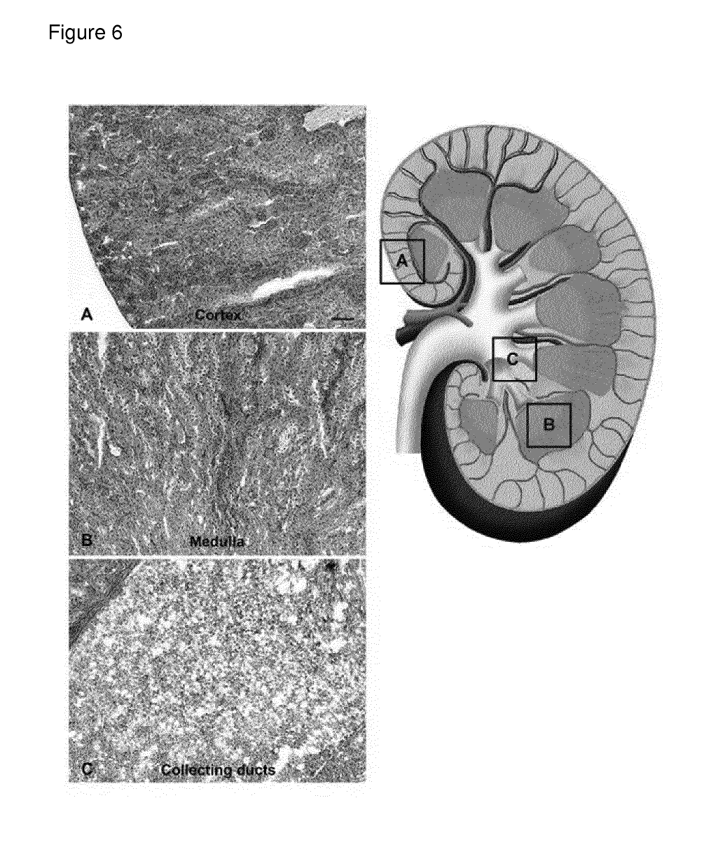

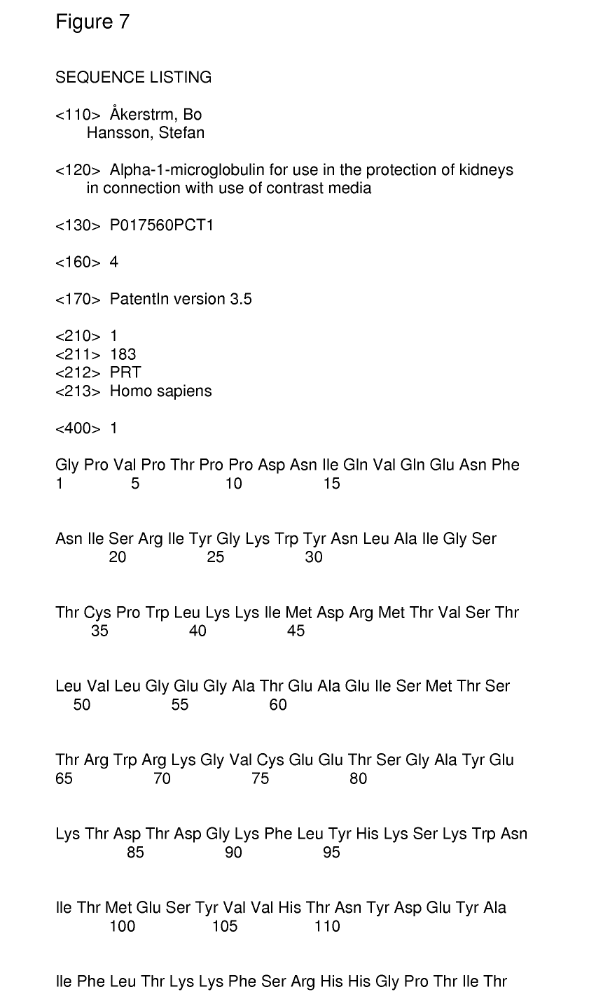

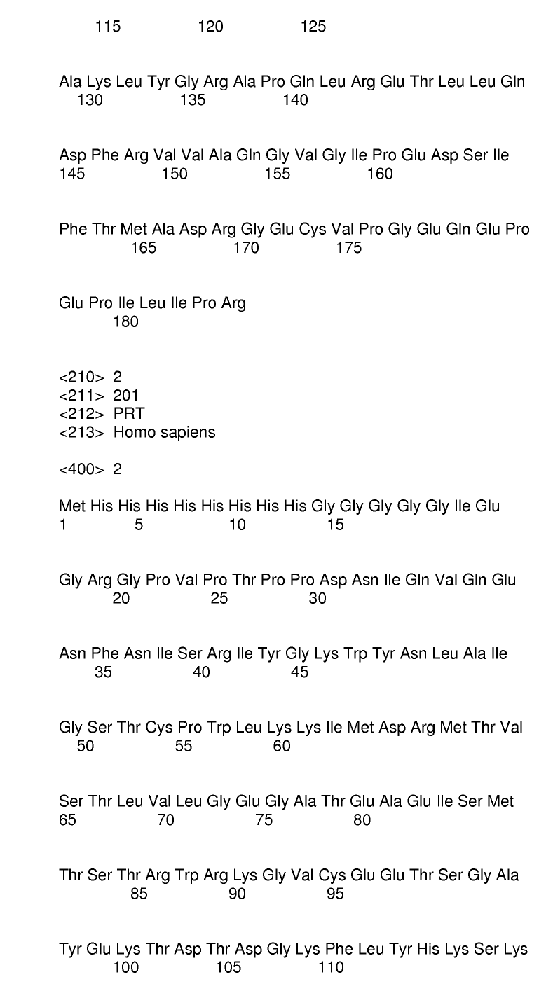

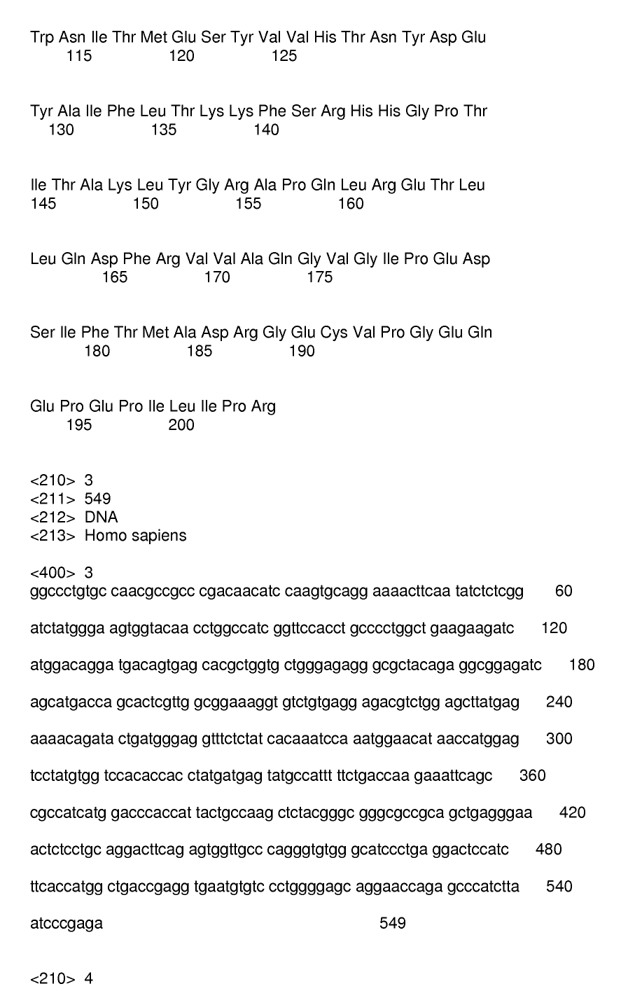

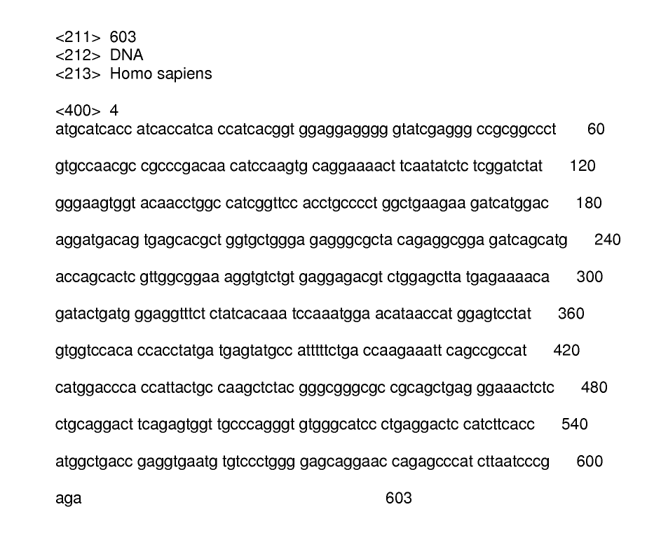

A qualitative SPECT/CT analysis was performed for ¹²⁵I-A1M and visualizes the activity distribution in the kidneys. The SPECT/CT images in FIG. 5 demonstrate a high uptake in the kidneys. ¹²⁵I-A1M (FIGS. 6 C and D) seems to localize in the kidney cortex. A slight uptake of ¹²⁵I-A1M in the thyroids can be seen as well.

IHC microscopical analysis (FIG. 6) shows that the infused A1M is mainly localized to the kidney cortex with gradually decreasing immunoreactivity towards the medulla and collecting ducts. Strong immunostaining of A1M can mainly be seen in the proximal tubular structures and subsets of glomeruli.

1-16. (canceled)

17. A method for treating or reducing the risks of side effects of medical contrast media, comprising administering an effective amount of alpha-1-microglobulin (A1M) to a subject in need thereof.

18. The method of claim 17, wherein the side effects comprise kidney-associated side effects.

19. The method of claim 17, wherein the side effects comprise medical contrast media-induced nephropathy (CIN).

20. The method of claim 17, wherein the side effects comprise oxidative stress.

21. The method of claim 17, wherein the subject exhibits one or more of the following risk factors:
age>75 years,
chronic kidney disease (CKD),
diabetes mellitus,
hypertension,
metabolic syndrome,
anemia,
multiple myeloma,
hypoalbuminemia,
renal transplant, and
hypovolemia and decreased effective circulating volumes as evidenced by one or more of congestive heart failure (CHF), ejection fraction (EF) of less than 40%, hypotension, and intra-aortic balloon counterpulsation.

22. The method of claim 17, wherein the subject exhibits one or more of the following risk factors: CKD and diabetes mellitus.

23. The method of claim 17, wherein the A1M is administered prior to, essentially at the same time as, during, or after administration of a medical contrast medium.

24. The method of claim 17, wherein the A1M is administered at the latest 24 hours before administration of a medical contrast medium.

25. The method of claim 17, wherein the A1M is administered at the most 1 hour before a medical contrast medium is administered and not later than concomitant with the medical contrast medium.

26. The method of claim 17, wherein the A1M is administered when a reliable marker for kidney function indicates kidney damage.

27. The method of claim 17, wherein the A1M is administered within a time period of from about 12 to about 48 hours after administration of a medical contrast medium.

28. The method of claim 17, wherein the A1M has at least 80% sequence identity to SEQ ID NO:1 or SEQ ID NO:2.

29. The method of claim 29, wherein the A1M has an amino acid sequence having the following amino acid residues at the following positions corresponding to the amino acid positions of human wildtype A1M set forth in SEQ ID NO:1:
Y22
C34
K69
K92
K118
K130
Y132
L180
I181
P182 and
R183.

30. A kit comprising (a) alpha-1-microglobulin (AIM) and (ii) a medical contrast medium.

31. The kit of claim 31, wherein the A1M is provided in a pharmaceutical composition.

32. The kit of claim 31, further containing instructions for use in a method for treating or reducing the risks of side effects of medical contrast media in a subject in need thereof.

33. The kit of claim 31, wherein the A1M has at least 80% sequence identity to SEQ ID NO:1 or SEQ ID NO:2.

34. The kit of claim 34, wherein the A1M has an amino acid sequence having the following amino acid residues at the following positions corresponding to the amino acid positions of human wildtype A1M set forth in SEQ ID NO:1:
Y22
C34
K69
K92
K118
K130
Y132
L180
I181
P182 and
R183.
